(19) United States
(12) Patent Application Publication
(10) Pub. No.: US 2019/0371440 A1
(43) Pub. Date: Dec. 5, 2019

(54) METHOD AND SYSTEM FOR HEALTH INFORMATION REPORTING

(71) Applicant: KONINKLIJKE PHILIPS N.V., EINDHOVEN (NL)

(72) Inventors: SAMAN PARVANEH, DANVERS, MA (US); MLADEN MILOSEVIC, STONEHAM, MA (US); DANIEL JASON SCHULMAN, JAMAICA PLAINS, MA (US); PORTIA E. SINGH, EVERETT, MA (US); AYMAN ALALAO, CAMBRIDGE, MA (US); MARK SABALAUSKAS, FRAMINGHAM, MA (US)

(21) Appl. No.: 16/480,176

(22) PCT Filed: Jan. 23, 2018
(86) PCT No.: PCT/EP2018/051544

§ 371 Date: Jul. 23, 2019

Related U.S. Application Data
(60) Provisional application No. 62/449,153, filed on Jan. 23, 2017.

(51) Int. Cl. G16H 10/60; H04W 4/02; G06F 21/62; G16H 15/00; A61B 5/00; G06F 21/34

(52) U.S. Cl. CPC G16H 10/60; H04W 4/025; G06F 21/34; G16H 15/00; A61B 5/6801; G06F 21/6245

(57) ABSTRACT

A method (100) for providing a (210) report about an individual (210) comprising the steps of: (i) providing (110) a report generation system (200) comprising a processor (252); (ii) receiving (120), by the processor, a command to generate a report comprising demographic information, medical/health information, and/or contextual information, where the contextual information comprises information, other than medical information, impacting the health or care of the individual; (iii) collecting (130), by the processor, medical information about the individual; (iv) collecting (140), by the processor, contextual information; (v) generating (150), by the processor, a report with the demographic information, medical/health information, and/or the contextual information; and (vi) communicating (160) the generated report to the individual or an intended recipient (280).

### CROSS-REFERENCE TO RELATED APPLICATIONS

The present disclosure claims priority to and benefit of U.S. Provisional Application No. 62/449,153, filed Jan. 23, 2017, the entirety of which is hereby incorporated by reference herein.

### FIELD

The present disclosure is directed generally to methods and systems for quickly gathering and collating demographic, medical/health, and contextual data about an individual into a report to provide to healthcare providers.

### BACKGROUND

According to the Centers for Disease Control and Prevention, there were more than 135 million visits to hospital Emergency Departments (ED) in the United States in 2011, resulting in hospital admission in more than 16 million of those visits. Given the vast number of Emergency Department visits, it is important that hospitals and healthcare providers have ready access to patient data. However, according to some estimates, significant gaps in information exchange, including communication of incorrect and incomplete information to ED staff, occur with almost one-third of ED visitors. The information deficiency may be even more common with sicker patients, thereby resulting in prolonged hospital stays.

Lack of critical patient health information such as baseline functional status can lead to fragmented and inefficient care, and thus can have a significant impact on the quality of emergency care. It is estimated that patients associated with an information gap experience ED stays of an additional 1.2 hours compared to other patients, which leads to patient dissatisfaction, overcrowding, and potentially to decreased quality of patient care.

In addition to gaps in patient health information, there is also a lack of contextual information that would further benefit patient care. Contextual information includes any environmental, demographic, historical, and/or society information, for example, that could impact the health and/or care of a patient. As just one example, air quality may impact patients with respiratory ailments or disease.

There has been a recent trend in consumer technology toward increasing collection of health information and the provision of health and safety-related services and information through personal and in-home devices. However, most efforts are consumer-directed, having little contact with the healthcare system. Accordingly, there are few systems for quickly collecting health information from diverse sources and formatting that information into a structured and clinically meaningful format for healthcare providers, particularly in emergency situations.

### SUMMARY

In view of the foregoing, it would be beneficial to provide methods and systems that gather and collate demographic, health, and contextual information about an individual into a clinically meaningful format for healthcare providers.

Accordingly, the present disclosure is directed to methods and systems for generating a report for patients and/or healthy individuals. Various embodiments and implementations herein are directed to a device or system that receives health information about an individual from one or more sources, including databases of electronic health records, wearable and in-home monitoring devices, and many other sources. The system also receives contextual information that could impact the health or care of an individual. A structured and clinically meaningful report is generated that combines both the gathered health information and contextual information, and that report is provided to relevant parties such as health care providers through an appropriate medium such as a website, smartphone, printed report, and other mediums. The collection of information and generation of a report can be triggered by any of a variety of events, including transport to an emergency department, an event or activity detected by in-home and wearable sensors, and many other events.

Generally in one aspect, a method for generating a report is provided. The method includes the steps of: (i) providing a report generation system; (ii) receiving a command to generate a report including medical information and contextual information about an individual, where the contextual information includes information, other than medical information, impacting the health or care of the individual; (iii) collecting medical information about the individual; (iv) collecting contextual information; (v) generating a report including the medical information and the contextual information; and (vi) communicating the generated report to the individual or an intended recipient.

According to an embodiment, the method further includes the step of storing medication information about the individual.

According to an embodiment, the method further includes the step of storing the medical information.

According to an embodiment, the method further includes the step of storing contextual information.

According to an embodiment, the method further includes the step of prompting the individual for authorization to communicate the generated report.

According to an embodiment, the method further includes the step of notifying the individual or the intended recipient that the generated report is available.

According to an embodiment, the method further includes the step of notifying the individual or the intended recipient that the generated report was accessed.

According to an embodiment, the method further includes the step of revoking access to the generated report.

According to an embodiment, the medical information is collected from an electronic health record.

According to an embodiment, the medical information is collected from a wearable device or an in-home monitoring device.

According to an embodiment, the contextual information includes information about the individual's environment.

According to an embodiment, the command to generate a report is created automatically following a predetermined trigging event.

According to an aspect is a system for providing a report. The system includes: a health record database; a contextual information module; and a processor configured to: (i) collect, from the health record database, medical information about the individual; (ii) collect, from the contextual information module, contextual information; (iii) generate a report including the medical information and the contextual information; and (iv) communicate the generated report to the individual or an intended recipient.

According to an embodiment, the system further includes at least a wearable device and/or an in-home monitoring device (such as a motion sensor for in-home monitoring of ADLs), and the contextual and/or medical information includes data from the wearable device and/or in-home monitoring device.

According to an embodiment, the system collects, directly from the wearable device, an in-home monitoring device, and/or a backend database, the medical information about the individual.

According to an aspect is a device for generating a report. The device includes: a communications module, and a processor configured to: (i) collect, via the communications module, medical information about the individual from a health record database; (ii) collect, via the communications module, contextual information from a contextual information module; (iii) generate a report including the medical information and the contextual information; and (iv) communicate the generated report to the individual or an intended recipient.

According to an embodiment, the processor is further configured to collect, via the communications module, medical information about the individual and/or contextual information about the individual from a wearable device and/or an in-home monitoring device.

According to an embodiment, the processor is further configured to notify the individual, the intended recipient, a medical insurer, or other recipient that the generated report has been accessed.

Various embodiments relate to method (and related system for performing the method and non-transitory machine-readable storage medium encoded with instructions for performing the method) for sharing a report about an individual, the method including: receiving, by a processor of a report generator system, consumer data from at least one consumer device of an individual; receiving a trigger message including an identifier of the individual and an indication of the occurrence of a trigger event; generating a medical report based on the consumer data from the consumer device; in response to the message indicating the occurrence of the trigger event, configuring a webserver of the report generator system to provide access to the medical report associated with the individual; receiving a web request from a requestor device for retrieval of the medical report; providing the medical report to the requestor device.

Various embodiments additionally include determining whether the requestor device is located in the vicinity of a known hospital location; and denying access to the medical report when it is determined that the requestor device is not located in the vicinity of a known hospital location, wherein providing the medical report is conditional on determining that the requestor device.

Various embodiments are described wherein the step of determining whether the requestor device is located in the vicinity of a known hospital location includes determining whether a source network address of the message is a network address associated with a known hospital network.

Various embodiments are described wherein the step of generating a medical report is performed prior to receiving the trigger message.

Various embodiments are described wherein the step of configuring a webserver of the report generator system to provide access to the medical report associated with the individual includes configuring a uniform resource locator (URL) to point to the medical report, wherein prior to the step of configuring, the URL is configured to point to a resource other than the medical report.

Various embodiments are described wherein the URL is encoded into a QR code that is at least one of attached to and displayed on the at least one consumer device.

Various embodiments are described wherein the trigger event includes the individual pressing a button of the at least one consumer device.

Various embodiments are described wherein the web request is received as part of the trigger message.

Various embodiments additionally include, in response to receiving the web request: transmitting an authorization request to at least one of the individual and a caregiver of the individual; receiving an authorization response; and denying access to the medical report when the authorization response indicates that access should be denied, wherein providing the medical report is conditional on the authorization response indicating that access should be allowed.

Various embodiments additionally include wherein the consumer device is a wearable device that gathers at least one of vital sign data and activity data related to the individual.

As used herein for purposes of the present disclosure, the term “processor” is used generally to describe various apparatus components relating to the operation of the recommendation apparatus, system, or method. A processor can be implemented in numerous ways (e.g., such as with dedicated hardware) to perform various functions discussed herein. A “processor” can employ one or more microprocessors that may be programmed using software (e.g., microcode) to perform various functions discussed herein. A processor may also be implemented as a combination of dedicated hardware to perform some functions. Examples of processor components that may be employed in various embodiments of the present disclosure include, but are not limited to, conventional microprocessors, application specific integrated circuits (ASICs), and field-programmable gate arrays (FPGAs).

In various implementations, a processor may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects discussed herein. The terms “program” or “computer program” are used herein in a generic sense to refer to any type of computer code (e.g., software or microcode) that can be employed to program one or more processors or controllers. As used herein, the term “non-transitory machine-readable medium” will be understood to encompass both volatile and non-volatile memories, but to exclude transitory signals.

The term “user interface” as used herein refers to an interface between a human user or operator and one or more devices that enables communication between the user and the device(s). Examples of user interfaces that may be employed in various implementations of the present disclosure include, but are not limited to, switches, potentiometers, buttons, dials, sliders, track balls, display screens, various types of graphical user interfaces (GUIs), touch screens, microphones and other types of sensors that may receive some form of human-generated stimulus and generate a signal in response thereto.

Various embodiments may further include non-transitory computer-readable storage media, having embodied thereon a firewall program executable by a processor to perform methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles elucidated herein.

FIG. 1 is a flowchart of a method for providing demographic, medical, health and/or contextual information in a report, in accordance with an embodiment.

FIG. 2 is a schematic representation of a report generation system, in accordance with an embodiment.

### DETAILED DESCRIPTION OF EMBODIMENTS

The present disclosure describes various embodiments of a method and system for reporting health information to a healthcare provider. More generally, Applicant has recognized and appreciated that it would be beneficial to provide a method or system for gathering and collating relevant medical/health information into a clinically meaningful format for healthcare providers without disrupting medical workflow. A particular goal of utilization of certain embodiments of the present disclosure is to provide a reporting system that collates both health and contextual information to improve health care and emergency care.

In view of the foregoing, various embodiments and implementations are directed to a method and system that receives demographic and medical/health information about an individual from one or more sources, including databases of electronic health records, wearable devices, and/or in-home monitoring devices, among other sources. The system also receives contextual information that could impact the health or care of the individual. In addition, the system can receive demographic information about the individual, as well as information about advance directives and other short-term or long-term care planning. A structured and clinically meaningful report is generated that combines the gathered demographic information, medical/health information, and contextual information, and that report is provided to relevant parties such as health care providers through an appropriate medium such as a website, smartphone, printed report, and other mediums. The collection of information and generation of a report can be triggered by any of a variety of events, including an emergency department stay, activity detected by in-home and wearable sensors, and many other events.

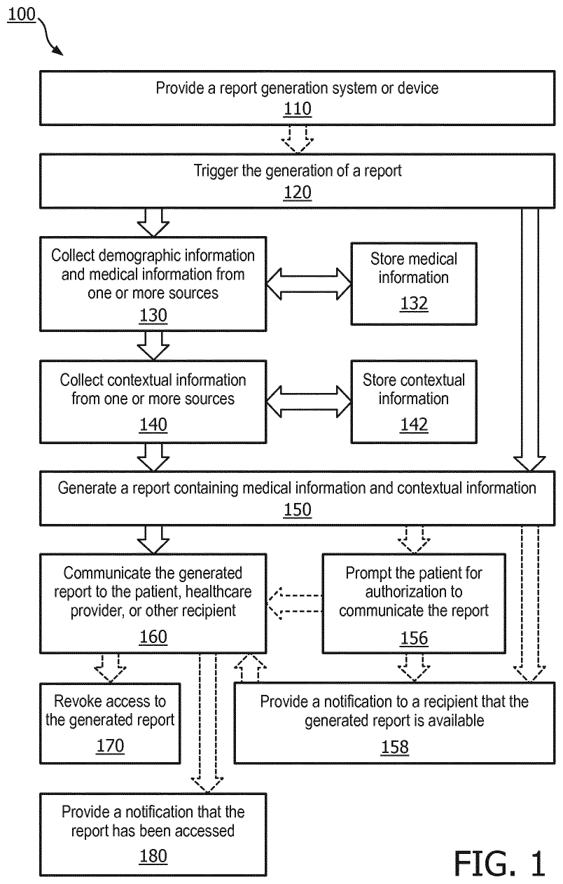

Referring to FIG. 1, in one embodiment, is a method 100 for reporting health and contextual information. The report may be generated for anyone, including any individual or subject in any setting or with a condition, emergency, wound, or other situation that may suggest or indicate a need for medical information either now or in the near future. In addition, the report may be generated for health individuals. The most common settings would be emergency rooms, field hospitals, emergency transportation vehicles, and similar locations, among many other possible settings.

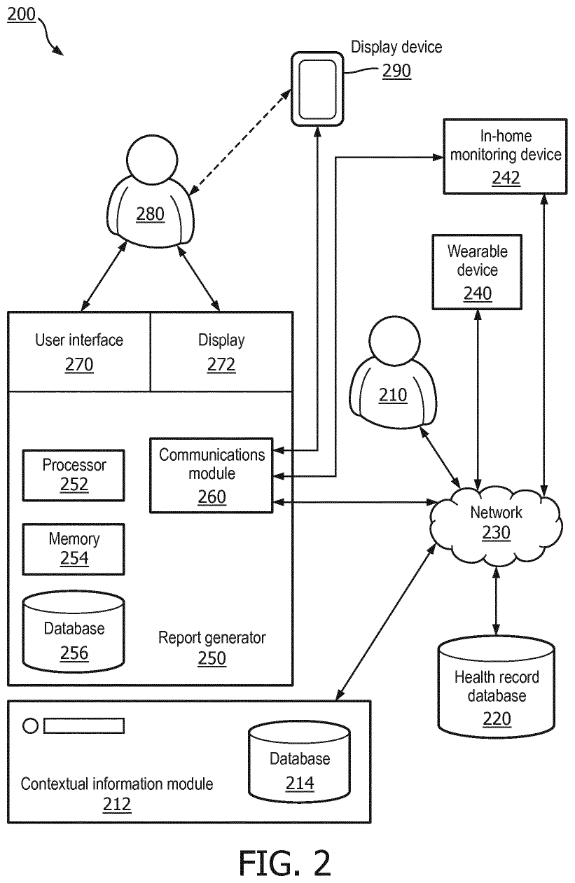

At optional step 110 of the method, a report generation system or device is provided. The report generation system or device may be any of the systems described or otherwise envisioned herein, including but not limited to system 200 in FIG. 2. According to another embodiment, the report generation system or device may already be implemented, installed, or existing at a local or remote location. For example, the report generation system or device may be implemented on a local or remote server or computer, among many other implementations.

At step 120 of the method, generation of a report by the report generation system or device is triggered. The generation of a report can be triggered by any number of events, including passive and/or active triggering events. According to an embodiment, generation of a report can be triggered via a Personal Emergency Response System (PERS), a wearable sensor, and/or via a user interface of a smartphone or computer app. The PERS device or wearable sensor, for example, might be continuously or periodically monitoring for an automatic triggering event, or might include an activation mechanism such as a button or user interface, which might require a tactic activation event, a password, code, and/or biometric activation.

The action by the individual can, for example, automatically grant permission to the system to access demographic information, health/medical information, and/or contextual information for generation of a report. For example, an individual wearing an alert or monitoring device can trigger generation of a report in response to an event such as a fall, a doctor's visit, an emergency department visit, or other events. Alternatively, the alert or monitoring device may automatically trigger generation of a report in response to an event that would also trigger an alert, such as a fall. In addition to the individual, the report may be triggered by an informal caregiver such as a family member, friend, or neighbor, anyone else granted permission by the individual to access and/or transfer the information or report, such as a home health aide, designated surrogate listed in an advance care directive, or an emergency caregiver such as an emergency medical responder, law enforcement officer, or a firefighter, a healthcare professional, or any of a variety of other individuals.

According to another embodiment, generation of a report can be triggered by an operator or agent of the alert or monitoring device when an adverse event is identified. The subject may therefore provide advance authorization to the monitoring company for report generation whenever an adverse event is identified. Any advance authorization may include, for example, a list of specific individuals and/or categories or classes of individuals that may have access to the generated report. For example, a subject may fall in their home and activate a monitoring device and/or wearable device that they wear around their neck, on their wrist, in their clothing, or elsewhere. Activation of the device triggers a notification to an emergency responder, firefighter, and/or law enforcement officer, among other possibilities, who will assist the individual in their current situation. In addition, activation of the device may automatically trigger generation of a report. Alternatively, the subject may be prompted to trigger generation of a report after triggering the emergency notification.

According to another embodiment, a report can be triggered by scanning a QR code on an identification card, on a monitoring device, and/or on a wearable device that the individual wears around the neck, on the wrist, in the clothing, or elsewhere. The QR code directs the scanner to access a website, send a signal to a remote device or computer, or otherwise trigger the generation of a report. For example, the individual may enter the emergency department and enable the generation or access of a report by allowing the QR code to be scanned, which provides permission to the healthcare provider to get access to the generated report in the emergency situation. Similarly, a report can be triggered by detecting a passive or active signal from the monitoring device and/or wearable device, such as an RFID, Bluetooth, WiFi, and/or other signal.

According to another embodiment, a report can be triggered automatically when a certain medical condition is detected. For example, the individual or subject may be periodically or continuously monitored by a monitoring device and/or wearable device, and that device may detect a medical condition, anomaly, and/or exacerbation that automatically triggers the generation of a report. Notably, the detection of the subject's state may simultaneously trigger an emergency condition such as an ambulance, a call to a healthcare provider, or any other emergency situation activation. As one example, the device may be programmed to detect a coughing fit, wheezing, high or low blood pressure, high or low temperature, oxygen saturation, high or low heart rate, fluid balance, ECG variances, or any of a variety of other health conditions or traits. According to an embodiment, the subject may agree to this automatic triggering when accepting the terms of service of the company. The subject and/or the healthcare professional may determine the triggers that are programmed to activate report generation. The report may be automatically generated, or may be generated only after a prompt to the subject upon which the subject acts.

According to another embodiment, a report may be generated automatically when the individual is within a certain geographic location, or within a predetermined geofence. For example, the subject may have a wearable device, monitoring device, and/or smartphone that automatically monitors geographic location using a GPS sensor. The device may be programmed such that whenever the device is within or sufficiently close to a location, a report is automatically generated, and/or a prompt for report generation is provided to the subject. For example, the device may be programmed to generate a report or provide a report prompt to the subject whenever the device is located within a hospital. As another example, the wearable device, monitoring device, and/or smartphone that automatically monitors its location relative to the location of another device. For example, the system may include a report generation activator that has a geofence associated with it, and crossing that geofence will trigger a report or prompt. For example, ambulances and police cars may include or include a geofence that can trigger a report or prompt. As another example, a hospital may have a geofence that triggers generation of a report whenever the subject's device or the subject crosses the geofence.

At step 130 of the method, demographic information, health information, and/or medical information is gathered from one or more sources. For example, health or medical information can be gathered from a health database, a medical records database, a device database, or other databases. The individual may have, for example, medical records stored with an electronic health record provider or service. Health or medical information can also be gathered from wearable devices, such as activity levels, number of steps, heart rate, blood pressure, temperature, sleep quality, respiration rate, or a variety of other parameters or measurements as described or otherwise envisioned herein. According to an embodiment, health or medical information from the wearable devices may be gathered from the backend system of the devices, from a device database, and/or directly from the devices.

According to an embodiment, the health or medical information is continuously and/or periodically collected and stored for future use at step 132 of the method. Rather than only collecting health or medical information in response to a trigger, the system may gather health information continuously and/or periodically at all times so that the information is readily available, and so that there is data for a historical comparison or analysis. This continuously and/or periodically gathered health or medical information can be stored in a local or remote database such as a health database, a medical records database, or other databases.

At step 140 of the method, contextual information is gathered from one or more sources. For example, contextual information can be gathered from an online source, a third-party service provider, a website, a local or remote server or database, or from a variety of other sources. Contextual information may include, for example, weather data, air quality, or a variety of other information that could impact the health and/or care for the individual. As an example, the contextual information may be air quality, temperature, humidity, pollen counts, atmospheric pressure, sunlight levels, cloud cover, or a variety of other information that could impact the health and/or care for the individual. The contextual information may be current information, past information, or both. For example, the system may access a weather service that provides free or paid access to current and historical weather data for a variety of different locations. Accordingly, the system may request to download or otherwise retrieve the current weather condition at the individual's location, along with weather conditions at the individual's one or many locations in the previous 24 hour period, 48 hour period, or a variety of other time periods.

According to an embodiment, the contextual information is continuously and/or periodically collected, accessed, and/or stored for future use at step 132 of the method. Rather than only collecting contextual information in response to a trigger, the system may gather contextual information continuously and/or periodically at all times so that the information is readily available, and so that there is data for a historical comparison or analysis. This continuously and/or periodically gathered contextual information can be stored in a local or remote database.

At step 150 of the method, a report is generated. The report contains gathered health or medical information and gathered contextual information, and can also contain demographic information about the individual, and tracking or other in-house identification information for the individual, such as health insurance information, an identifier, or other tracking information. The report may be organized into a meaningful and easy-to-read and easy-to-understand format. The report may be based on a standard format, an individual-specific format, a location-specific format, a medical condition-format, or a variety of other formats.

According to an embodiment, the format of the report is at least partially dependent upon the intended or authorized recipient. For example, an authorized caregiver without a medical degree may receive a more simplified report, while a physician may receive a report with a greater amount of medical information and health records, including, for example, lab results. An emergency responder or emergency care provider may receive an abbreviated or otherwise modified report that provides the most essential information in the shortest format, to save time. Many other recipient-dependent formats and modifications of the report are possible.

At step 160 of the method, the report is communicated to the individual, healthcare provider, and/or other intended recipient. For example, according to an embodiment, the generated report is generated and is available for anyone to access, or is only available to individuals with authorization and/or a password. The recipient of the report may be emergency personnel, law enforcement, firefighters, healthcare professionals including nurses, physicians, and administrators, family members, caregivers, and any other individual capable and/or authorized to receive the report.

Given the potentially sensitive nature of the information in the generated report, it may be restricted such that it is only available to authorized individual. For example, the report may only be available through a secure link, website, or other portal, and will only be accessible with an access code, QR code, password, biometric parameter, and/or other access mechanism. The report may be automatically provided or uploaded to a caregiver portal, electronic health record database, healthcare professional portal, or other access site. According to another example, the generated report may be automatically delivered to a smartphone, portable computing device, laptop, desktop, or other computer or display via an app or other access. To protect the individual's privacy, the report can be disabled after it is accessed a certain number of times, after a specified time has elapsed from a trigger or from a first access, and/or based on one or more other thresholds or limitations.

According to another embodiment, the report will only be available if the individual has authorized communication to another party. Accordingly, at optional step 156 of the method, the system provides a prompt to the individual to authorize delivery of the report to an individual. The prompt may be substantially contemporaneous with the generation of the report, may be provided when the system or service is activated or modified by the individual, or may be provided after the report has been generated. For example, the individual's smartphone may include an app or other user interface that asks, when a report is generated in response to a trigger (which may be provided through the same app), whether and/or to whom to provide the report. The authorization may include a selection of specific individuals, a timeframe, a location, and/or one or more other variables or parameters.

According to another embodiment, the intended or authorized recipient of the generated report is notified that they have been authorized, and/or that the report is available for access or review. Accordingly, at optional step 158 of the method, the system provides a notification to the intended or authorized recipient of the generated report that it is available. This may be a text message, a smartphone notification, an email, a printout, or any of a variety of other notification methods.

The report can be accessed by, delivered to, or otherwise made available to the intended or authorized recipient in any of a variety of different ways. For example, the authorized recipient may log into a portal or website to access the report, which may or may not require a password, biometric authorization, or other security measure. As another example, the authorized recipient may receive or access a copy of the report via a smartphone app, a computer program, or another software package or application. As another example, the report may be printed or otherwise directly prepared or presented in a hardcopy form to the authorized recipient.

According to an embodiment, the individual may wear or possess a code such as a QR code, or a biometric parameter, or other authorization element that must be scanned or measured or otherwise provided in order to provide the access. For example, emergency responders assisting an individual in their home or in the hospital may be able to scan a QR code or other access code, such as a barcode on a medical bracelet, even if the individual is unconscious or unable to provide a verbal authorization.

According to an embodiment, the generated report may only be accessed when the intended or authorized recipient accesses the report through a pre-determined hospital IP address, when the location of the individual is determined by a GPS sensor, smartphone connection, WiFi connection, or other device such as a wearable device, and/or when the individual has authorized the access by physiological parameters such as ECG morphology, heart rate, or heart rate variability for user authentication. For example, the report may be generated and/or access may be granted if the location of the individual or authorized recipient is determined to be at the authorized location, such as within or close to a hospital.

Once the report is received, it may be utilized by the recipient in one or more of a wide variety of ways. For example, the report may be reviewed by emergency department personnel in order to obtain the medical history of the individual, the current health status of the individual including vital measurements, the possible cause for the visit, and/or the factors that may have triggered the adverse event or medical condition, among other information.

At optional step 170 of the method, access to the generated report is revoked, denied, or otherwise limited after a certain criterion or parameter, such as a predetermined timeframe. For example, the report may be removed, archived, or deleted automatically after a certain amount of time, after it has been accessed once or any other predetermined number of times, or after the individual has requested removal, archiving, or deletion, among other triggers. For example, the individual may determine that access to the report should no longer be available at all, or to certain other individuals.

At optional step 180 of the method, a notification is provided when the report is accessed or read. For example, if the report is accessed by the individual, a healthcare provider, and/or any authorized or intended recipient, the system can generate a notification that the report has been accessed. The notification may optionally include information about the access, including the identity or other information about the entity or person accessing the report, the time, date, location, or any other information about the access. The notification may be sent to the individual, the formal or informal caregiver, an insurance company, the healthcare professional, and/or to any designated or intended individual. The notification may be made through any mechanism, including but not limited to email, text message, smartphone notification, or any other type of notification.

Referring to FIG. 2, according to an embodiment, is a report generation device or system 200. The report generation device or system may be any of the systems described or otherwise envisioned herein. Report generation device or system 200 is associated with a subject 210. The individual or subject 210 can be anyone interested in monitoring health and providing health information to others via a generated report, including individuals with known medical conditions or individuals with no known health issues.

The report generation device or system 200 includes or is in communication with a health record database 220, which is a health database, a medical records database, or other database that stores health or medical information about the individual 210. The individual may have, for example, medical records stored with an electronic health record provider or service. The health or medical information can also be gathered from wearable devices, such as activity levels, number of steps, heart rate, blood pressure, temperature, sleep quality, respiration rate, or a variety of other parameters or measurements as described or otherwise envisioned herein.

The health record database 220 may be a local database which is a component of the report generation device or system 200, or it may be a remote database that is in communication with the report generation device or system 200. For example the system may include a wired and/or wireless network 230 that facilitates communication within the report generation device or system 200, and/or between the system and third-party entities, such as electronic health record databases, healthcare professionals, or other third parties.

According to an embodiment, report generation device or system 200 includes or is in communication with a wearable device 240. The wearable device may be a standard wearable device with a sensor, such as an activity monitor, or it may be a smartphone or other sensor intended to obtain sensor data directly from a wearer or user. The wearable device 240 may, for example, include a sensor configured to measure, determine, or derive blood pressure, heart rate, pulse, blood oxygen, body temperature, respiratory rate, heart sound, breathing sound, movement, skin moisture, or similar health measurements, other vital signs, or other parameters known in the art. In some embodiments, the wearable device 240 may provide identifications of activity or measures (e.g. accelerometer magnitude data) thereof. Accordingly, the wearable device may include a sphygmomanometer, heart rate monitor, pulse oximeter, thermometer, accelerometer, microphone, electromagnetic sensor, and/or any other type of sensor.

According to an embodiment, report generation device or system 200 includes or is in communication with an in-home monitoring device 242. In-home monitoring device 242 may be any device that monitors an individual or the individual's environment. For example, in-home monitoring device 242 may be a video device, a motion sensor, a proximity sensor, a sound monitor, or a variety of other devices or sensors. The device may be in communication with report generation device or system 200 directly, or may communicate with the device or system via network 230 or any other communication mechanism. The in-home monitoring device 242 may gather information such as, for example, identified activities of daily living or number of bathroom visits by the individual over a time period.

The report generation device or system 200 also includes a contextual information module 212, which may be a server, web portal, database such as contextual information database 214, and/or any other component, system, device, or access method to provide contextual information. For example, the contextual information module 212 may be an online source, a third-party service provider, a website, a local or remote server or database, or a variety of other sources. The contextual information module 212 may be local to or remote from the other elements of the report generation device or system 200. For example, the contextual information module 212 may be located within the house of the individual 210, such as a component of the wearable device 240, or may be a remote server accessed over the Internet. Although the contextual information module 212 in FIG. 2 is shown communicating with the network 230, it should be appreciated that any entity or component of the report generation device or system 200 may communicate with the contextual information module 212.

According to an embodiment, the contextual information module 212 provides contextual information such as, for example, weather data, air quality, or a variety of other information that could impact the health and/or care for the individual. As an example, the contextual information may be air quality, temperature, humidity, pollen counts, atmospheric pressure, sunlight levels, cloud cover, or a variety of other information that could impact the health and/or care for the individual. The contextual information may be current information, past information, or both. For example, the system may access a weather service that provides free or paid access to current and historical weather data for a variety of different locations. According to an embodiment, the processor can be configured to store or control the storage of contextual information in local or remote storage, including database 214, database 256, or any other storage device.

The report generation device or system 200 also includes a report generator 250. The report generator is a report generation engine, which combines demographic information, health data, and/or other medical information with contextual information to generate a meaningful, structured report. The report generator 250 can be a computer algorithm or program, an application on a smartphone or other portable computer, and/or a device. The report generator 250 receives input about an individual from multiple sources, local and/or remote, and collates that information into a report for healthcare providers or other authorized individuals.

According to an embodiment, the report generator 250 can include a processor 252 which is configured or programmed to facilitate the generation of a report. For example, the processor may be configured or programmed to receive input about an individual from multiple sources, local and/or remote, and collate that information into a report. The processor 252 may be programmed using software to perform various functions discussed herein, and can be utilized in combination with a memory 254 and/or database 256. Memory 254 and/or database 256 can store data, including one or more commands or software programs for execution by processor 252, as well as various types of data. For example, the memory 254 may include a non-transitory computer readable storage medium that includes a set of instructions that are executable by processor 252, and which cause the system to execute one or more of the steps of the methods described herein.

According to an embodiment, the report generator 250 can include a communications module 260 to facilitate wired and/or wireless communication between the report generator and other devices and/or networks, such as network 230. The communication module 260 may be facilitated through the use of one or more antennas, for example. The communication module 260 can facilitate communication with one or more networks or with other devices, for example, by using wireless methods that are known, including but not limited to Wi-Fi, Bluetooth, 3G, 4G, LTE, and/or ZigBee, among others.

The report generation device or system 200 may include a user interface 270 to receive input from an individual 210, and/or an emergency responder, healthcare provider, or other intended or authorized entity 280. The user interface may be a button or multiple buttons, a microphone, a key stroke input, a slider, a touchscreen, or any of a variety of other inputs. For example, individual 210 may utilize the user interface 270 to instruct or authorize the report generator 250 to create a report. As another example, the individual 210 may utilize the user interface 270 to program parameters for report generation and delivery, including triggering events or parameters, authorized recipients, geofences and locations, and any of the other parameters described or otherwise envisioned herein.

According to an embodiment, an individual 210 or another entity 280 may provide an input remotely via a computer which communicates the input to the device via network 230. For example, the user may utilize a computer or a wearable device 240 to program the system with parameters for report generation and delivery, or to authorize generation or delivery of a report.

The report generation device or system 200 may include a display 272 which is utilized to provide information such as a generated report to the individual 210 and/or to an emergency responder, healthcare provider, or other intended or authorized entity 280. The display may be, for example, an LED-based, LCD-based, or e-paper type display. In other embodiments, the display may be a touch screen display that allows the user to directly interact with the wearable device through physical contact and/or gestures.

According to an embodiment, an individual 210 or other intended or authorized entity 280 may receive or review the report using a display remote from the report generator 250 and/or remote from the report generation device or system 200. For example, the individual may review a report on their wearable device, on a computer, on their television, or using any other display. The intended or authorized entity 280 may review a generated report on their wearable device, on a computer, or using any other display.

According to an embodiment, system 200 can optionally include a display device 290, which may also serve as a user input device. The device 290 may be, for example, a smartphone or other portable device. According to yet another embodiment, the device 290 may be a computer such as a desktop, laptop, tablet, or other permanent or semi-permanent computing device. The device 290 may receive input from the healthcare provider or other intended or authorized entity 280, such as a request for a report. The device 290 may also display information to the healthcare provider or other intended or authorized entity 280, such as the generated report.

According to an embodiment, processor 252 of report generation device or system 200 is configured to execute one or more of the steps of the methods described herein. For example, the processor can be configured to gather, authorize, or direct the gathering of health or medical information is gathered from one or more sources, such as from a health record database 220, and/or other databases. Health or medical information can also be gathered from wearable device 240, such as activity levels, number of steps, heart rate, blood pressure, temperature, sleep quality, respiration rate, or a variety of other parameters or measurements as described or otherwise envisioned herein. According to an embodiment, the processor can be configured to store or control the storage of the gathered health or medical information in local or remote storage.

In some embodiments, consumer data is continually gathered and stored for an individual, e.g., in the database 256 of the report generator 250 or in some other storage (not shown) accessible to the processor of the report generator. For example, data from the wearable device 240 and in-home monitoring device 242 may be periodically uploaded for long term storage. The devices may be configured to push this information for storage, the report generator 250 may be configured to pull this information (such a configuration may not require any reconfiguration of the data producing devices 240, 242), or a combination of push and pull approaches may be used. For example, in-home monitoring device 242 may be configured to push its gathered data while the report generator 250 may be configured to pull data from the wearable device 240.

The processor 252 of report generation device or system 200 can also be configured to gather or direct the gathering of contextual information from one or more sources. For example, the processor can be configured or programmed to retrieve, request, query, obtain, or otherwise get contextual information from a contextual information module 212, which may be a server, web portal, database such as contextual information database 214, and/or any other component, system, device, or access method to provide contextual information. For example, the contextual information module 212 may be an online source, a third party service provider, a website, a local or remote server or database, or a variety of other sources. The contextual information module 212 may be local to or remote from the other elements of the report generation device or system 200.

The processor 252 of report generation device or system 200 can also be configured to generate a report from the gathered medical information and contextual information. For example, the processor can be configured or programmed to generate a report via report generator 250, as described or otherwise envisioned herein. The report contains gathered health or medical information and gathered contextual information, and can also include demographic information about the individual, and tracking or other in-house identification information for the individual, such as health insurance information, an individual identifier, or other tracking information. The report may be organized into a meaningful and easy-to-read and easy-to-understand format. The report may be based on a standard format, an individual-specific format, a location-specific format, a medical condition-format, or a variety of other formats.

In various embodiments, the medical information may be the information previously gathered from consumer devices such as the wearable device 240 and in-home monitoring device 242. As such, the report may be generated using information available from these devices, providing caregivers, first responders, clinicians, and other concerned parties access to relevant portions of the data these devices gather that wouldn't otherwise be captured by the individual's medical records.

The processor 252 of report generation device or system 200 can also be configured to communicate or cause communication of the generated report to the individual 210 and/or to the intended or authorized recipient 280. For example, the generated report may be communicated to emergency personnel, law enforcement, firefighters, healthcare professionals including nurses, physicians, and administrators, family members, caregivers, and any other individual capable and/or authorized to receive the report. The report may only be available through a secure link, website, or other portal, and will only be accessible with an access code, QR code, password, biometric parameter, and/or other access mechanism. The report may be automatically provided or uploaded to a caregiver portal, electronic health record database, healthcare professional portal, or other access site. According to another example, the generated report may be automatically delivered to a smartphone, portable computing device, laptop, desktop, or other computer or display via an app or other access.

For example, a QR code or other mechanism may encode a URL pointing to a website where the report will be hosted. In some embodiments, the URL may normally point to a blank page or otherwise a page that does not contain any health or other private information. Upon occurrence of a trigger and subsequent report generation, the server may make the report available at the URL. For example, the server may place the report in a storage location to which the URL already points or may reconfigure the web server software such that the URL is now interpreted to access a different storage location where the report will be located. In various alternative embodiments, a report may not be generated upon occurrence of a trigger and, instead, continuously curated. A trigger in such embodiments may cause the server to make the already-generated report available at the URL typed into a browser, encoded in the QR code, associated with a password or biometric parameter, or otherwise associated with another access mechanism.

According to an embodiment, the processor 252 of report generation device or system 200 can also be configured to provide or cause the provision of a prompt to the individual to authorize delivery of the report to an individual. For example, the individual's smartphone may include an app or other user interface that asks, when a report is generated in response to a trigger (which may be provided through the same app), whether and/or to whom to provide the report. The authorization may include a selection of specific individuals, a timeframe, a location, and/or one or more other variables or parameters. Many other methods of determining or confirming authorized delivery or receipt of the generated report are available.

According to an embodiment, the processor 252 of report generation device or system 200 can also be configured to provide or cause the provision of a notification to the individual or the intended or authorized recipient of the generated report that the generated report is available. This may be a text message, a smartphone notification, an email, a printout, or any of a variety of other notification methods. Many other methods of providing notifications to the individual or the intended or authorized recipient are available.

According to an embodiment, the processor 252 of report generation device or system 200 can also be configured to confirm access to the generated report by an intended or authorized recipient 280. For example, if the generated report is only be accessible upon entry of a code, password, or other information, or is only accessible at a certain location or through a pre-determined IP address, among many other possible restrictions, the processor 252 may be configured or programmed to receive access information such as the code, password, IP address, or GPS data. The processor 252 will be further configured or programmed to analyze the received access information, such as comparison to preprogrammed access data in a database, and will determine whether the individual or computer seeking access has authorization to do so.

According to an embodiment, the processor 252 of report generation device or system 200 can also be configured or programmed to revoke or control the revocation of the generated report. For example, the report may be removed, archived, or deleted automatically after a certain amount of time, after it has been accessed once or any other predetermined number of times, or after the individual has requested removal, archiving, or deletion, among other triggers. Many other methods of denying or revoking access to the generated report are available.

According to an embodiment, the processor 252 of report generation device or system 200 can also be configured or programmed to generate and/or provide a notification when the generated report is accessed or read. For example, if the report is accessed by the individual, a healthcare provider, and/or any authorized or intended recipient, the system can generate a notification that the report has been accessed. The notification may optionally include information about the access, including the identity or other information about the entity or person accessing the report, the time, date, location, or any other information about the access. The notification may be sent to the individual, the formal or informal caregiver, an insurance company, the healthcare professional, and/or to any designated or intended individual. The notification may be made through any mechanism, including but not limited to email, text message, smartphone notification, or any other type of notification.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method comprising:
receiving, via an input interface, trigger data comprising a patient identifier and an indication of a trigger event;
generating, in response to the received trigger data, a trigger data-specific medical report based on the received trigger data and patient data from a patient device associated with the patient;
configuring, via an output interface and in response to the generated trigger data-specific medical report, a webserver to provide access to the trigger data-specific medical report;
receiving, via the input interface, web request data associated with a request to access the trigger data-specific medical report via the configured webserver; and
providing, via the output interface, the trigger data-specific medical report based on the web request data
wherein the trigger data-specific medical report is associated with known medical location data, the web request data comprises requester location data, and the step of providing the trigger data-specific medical report is conditioned upon the known medical location data and the requester location data, and wherein the trigger data-specific medical report is provided when the requester location is within the known medical location data.

2.-3.(canceled)

4. The method of claim 1, wherein the requester location data comprises a source network address associated with the request, and wherein the known medical location data comprises at least one network address associated with a known hospital network.

5. The method of claim 1, wherein a preliminary medical report is generated prior to receiving the trigger message and wherein the trigger data-specific medical report generated in response to the trigger message is an updated medical report.

6.-7. (canceled)

8. The method of claim 1, wherein the trigger event comprises the individual pressing a button associate with the patient device.

9. The method of claim 1, wherein the trigger event comprises entering a location associated with a known medical facility, and wherein the indication of the trigger event comprises location data associated with a known medical facility.

10. The method of claim 1, further comprising, in response to receiving the web request:
transmitting an authorization request to at least one of: the patient and a caregiver of the patient;
receiving an authorization response; and
providing the medical report when the authorization response indicating that access should be allowed.

11. The method of claim 1, wherein the patient device is at least one of: a wearable device that gathers at least one of vital sign data and activity data, and an in-home monitoring device that gathers at least one of motion data and medication data.

12. The method of claim 1, wherein providing, via the output interface, the trigger data-specific medical report based on the web request data comprises providing a requester-specific format of the trigger data-specific medical report data.

13. The method of claim 1, wherein providing, via the output interface, the trigger data-specific medical report is conditioned upon an access threshold, wherein an access threshold comprises at least one of: a number of times accessed, a length of time from receipt of trigger data, and a length of time from first access.

14. A system comprising:
an input/output interface;
a storage device for storing patient data from at least one patient device associated with a patient; and
at least one processor configured to:
receive, via the input/output interface, trigger data comprising a patient identifier and an indication of a trigger event;
generate, in response to the received trigger data, a trigger data-specific medical report based on the received trigger data and patient data;
configure, via the input/output interface and in response to the generated trigger data-specific medical report, a webserver to provide access to the trigger data-specific medical report;
receive, via the input/output interface, web request data associated with a request to access the trigger data-specific medical report via the configured webserver; and
provide, via the input/output interface, the trigger data-specific medical report based on the web request data wherein the trigger data-specific medical report is associated with known medical location data, the web request data comprises requester location data, and the step of providing the trigger data-specific medical report is conditioned upon the known medical location data and the requester location data
and wherein the trigger data-specific medical report is provided when the requester location is within the known medical location data.

15.-16. (canceled)

17. The system of claim 14, wherein the requester location data comprises a source network address associated with the request, and wherein the known medical location data comprises at least one network address associated with a known hospital network.

18. The system of claim 14, wherein a preliminary medical report is generated prior to receiving the trigger message and wherein the trigger data-specific medical report generated in response to the trigger message is an updated medical report.

19.-20. (canceled)

21. The system of claim 14, wherein the trigger event comprises the individual pressing a button associate with the patient device.

22. The system of claim 14, wherein the trigger event comprises entering a location associated with a known medical facility, and wherein the indication of the trigger event comprises location data associated with a known medical facility.

23.-28. (canceled)
